(19) United States
(12) Patent Application Publication
(10) Pub. No.: US 2009/0081277 A1
(43) Pub. Date: Mar. 26, 2009

(54) PHARMACEUTICAL FORMULATIONS AND METHODS FOR TREATING OCULAR CONDITIONS

(75) Inventors: Michael R. Robinson, Irvine, CA (US); Patrick M. Hughes, Aliso Viejo, CA (US); Wendy M. Blanda, Tustin, CA (US); Scott M. Whitcup, Laguna Hills, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/859,310

(22) Filed: Sep. 21, 2007

(51) Int. Cl. A61K 47/48

(52) U.S. Cl. 424/428

(57) ABSTRACT

Biodegradable drug delivery systems suitable for injection into an ocular region or site and methods for treating ocular conditions. The drug delivery systems provide increased drug residency time and attendant therapeutic benefit.

### BACKGROUND

The present invention relates to formulations (drug delivery systems) and methods for treating ocular conditions. In particular the present invention relates to pharmaceutical formulations and methods for treating posterior ocular conditions by administering to an anterior ocular location a drug delivery system comprising a therapeutic agent (a drug) and a bioerodible polymer.

An ocular condition can include a disease, aliment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. A front of the eye or anterior ocular condition is a disease, ailment or condition which affects or which involves an ocular region or site, such as a periocular muscle, an eye lid or an eye ball tissue or fluid which is located anterior to the posterior wall of the lens capsule or ciliary muscles. Thus, a front of the eye ocular condition primarily affects or involves, the conjunctiva, the cornea, the conjunctiva, the anterior chamber, the iris, the posterior chamber (behind the iris but in front of the posterior wall of the lens capsule), the lens and the lens capsule as well as blood vessels, lymphatics and nerves which vascularize, maintain or innervate an anterior ocular region or site.

A front of the eye (anterior) ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can be considered to be a front of the eye ocular condition because a clinical goal of glaucoma treatment can be to reduce a hypertension of aqueous fluid in the anterior chamber of the eye (i.e. reduce intraocular pressure).

A posterior (or back of the eye) ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can also be considered a posterior ocular condition because a therapeutic goal of glaucoma treatment is to prevent the loss of or reduce the occurrence of loss of vision due to damage to or loss of retinal cells or optic nerve cells (i.e. neuroprotection).

Macular degeneration, such as age related macular degeneration (“AMD”) is a leading cause of blindness in the world. It is estimated that thirteen million Americans have evidence of macular degeneration. Macular degeneration results in a break down the macula, the light-sensitive part of the retina responsible for the sharp, direct vision needed to read or drive. Central vision is especially affected. Macular degeneration is diagnosed as either dry (atrophic) or wet (exudative). The dry form of macular degeneration is more common than the wet form of macular degeneration, with about 90% of AMD patients being diagnosed with dry AMD. The wet form of the disease usually leads to more serious vision loss. Macular degeneration can produce a slow or sudden painless loss of vision. The cause of macular degeneration is not clear. The dry form of AMD may result from the aging and thinning of macular tissues, depositing of pigment in the macula, or a combination of the two processes. With wet AMD, new blood vessels grow beneath the retina and leak blood and fluid. This leakage causes retinal cells to die and creates blind spots in central vision.

Macular edema (“ME”) can result in a swelling of the macula. The edema is caused by fluid leaking from retinal blood vessels. Blood leaks out of the weak vessel walls into a very small area of the macula which is rich in cones, the nerve endings that detect color and from which daytime vision depends. Blurring then occurs in the middle or just to the side of the central visual field. Visual loss can progress over a period of months. Retinal blood vessel obstruction, eye inflammation, and age-related macular degeneration have all been associated with macular edema. The macula may also be affected by swelling following cataract extraction. Symptoms of ME include blurred central vision, distorted vision, vision tinted pink and light sensitivity. Causes of ME can include retinal vein occlusion, macular degeneration, diabetic macular leakage, eye inflammation, idiopathic central serous chorioretinopathy, anterior or posterior uveitis, pars planitis, retinitis pigmentosa, radiation retinopathy, posterior vitreous detachment, epiretinal membrane formation, idiopathic juxtafoveal retinal telangiectasia, Nd:YAG capsulotomy or iridotomy. Some patients with ME may have a history of use of topical epinephrine or prostaglandin analogs for glaucoma. The first line of treatment for ME is typically anti-inflammatory drops topically applied.

Diabetic retinopathy is the leading cause of blindness among adults aged 20 to 74 years. Macular ischemia is a major cause of irreversible vision acuity loss and decreased contrast sensitivity in patients with diabetic retinopathy. The capillary nonperfusion and decreased capillary blood flow that is responsible for this ischemia is seen clinically on the fluorescein angiogram as an increase in the foveal avascular zone (FAZ) or an irregularity of the outline of the FAZ. These findings are predictors of the other, perhaps more well-known, sight-threatening complications of diabetic retinopathy, including macular edema and proliferative retinopathy. Perhaps more importantly, extensive capillary nonperfusion is also a predictor of a poor visual prognosis from diabetic retinopathy.

There are treatments available or in development for macular edema and proliferative retinopathy, such as laser photocoagulation, intravitreal corticosteroids and anti-VEGF therapies. Although laser photocoagulation has been studied for vision loss directly associated with macular ischemia, there is currently no known treatment for this indication.

The exterior surface of the normal globe mammalian eye has a layer of tissue known as conjunctival epithelium, under which is a layer of tissue called Tenon's fascia (also called conjunctival stroma). The extent of the Tenon's fascia extending backwards across the globe forms a fascial sheath known as Tenon's capsule. Under Tenon's fascia is the episclera. Collectively, the conjunctival epithelium and the Tenon's fascia is referred to as the conjunctiva. As noted, under Tenon's fascia is the episclera, underneath which lies the sclera, followed by the choroid. Most of the lymphatic vessels and their associated drainage system, which is very efficient at removing therapeutic agents placed in their vicinity, is present in the conjunctiva of the eye.

A therapeutic agent can be administered to the eye to treat an ocular condition. For example the target tissue for an antihypertensive therapeutic agent to treat the elevated intraocular pressure characteristic of glaucoma can be the ciliary body and/or the trabecular meshwork. Unfortunately, administration of an ocular topical antihypertensive pharmaceutical in the form of eye drops can result in a rapid wash out of most if not all of the therapeutic agent before it reaches the ciliary body and/or the trabecular meshwork target tissue, thereby requiring frequent redosing to effectively treat a hypertensive condition. Additionally, side effects to patients from topical administration of antiglaucoma medications and their preservatives range from ocular discomfort to sight-threatening alterations of the ocular surface, including conjunctival hyperemia (eye redness), stinging, pain, decreased tear production and function, decreased tear film stability, superficial punctate keratitis, squamous cell metaplasia, and changes in cell morphology. These adverse effects of topical antiglaucoma eyedrops can interfere with the treatment of glaucoma by discouraging patient dosing compliance, and as well long-term treatment with eyedrops is associated with a higher failure of filtration surgery. Asbell P.A., et al Effects of topical antiglaucoma medications on the ocular surface, Ocul Surf 2005 Jan.; 3(1):27-40; Mueller M., et al. Tear film break up time and Schirmer test after different antiglaucomatous medications, Invest Opthalmol Vis Sci 2000 Mar. 15; 41(4):S283.

It is known to administer a drug depot to the posterior (i.e. near the macula) sub-Tenon space. See eg column 4 of U.S. Pat. No. 6,413,245. Additionally, it is known to administer a polylactic implant to the sub-tenon space or to a suprachoroidal location. See eg published U.S. Pat. No. 5,264,188 and published U.S. patent application 20050244463

An anti-inflammatory (i.e. immunosuppressive) agent can be used for the treatment of an ocular condition, such as a posterior ocular condition, which involves inflammation, such as an uveitis or macula edema. Thus, topical or oral glucocorticoids have been used to treat uveitis. A major problem with topical and oral drug administration is the inability of the drug to achieve an adequate (i.e. therapeutic) intraocular concentration. See e.g. Bloch-Michel E. (1992). Opening address: intermediate uveitis, In Intermediate Uveitis, Dev. Opthalmol, W. R. F. Böke et al. editors, Basel: Karger, 23:1-2; Pinar, V., et al. (1997). Intraocular inflammation and uveitis” In Basic and Clinical Science Course. Section 9 (1997-1998) San Francisco: American Academy of Opthalmology, pp. 57-80, 102-103, 152-156; Böke, W. (1992). Clinical picture of intermediate uveitis, In Intermediate Uveitis, Dev. Opthalmol. W. R. F. Boke et al. editors, Basel: Karger, 23:20-7; and Cheng C-K et al. (1995), Intravitreal sustained-release dexamethasone device in the treatment of experimental uveitis, Invest. Opthalmol. Vis. Sci. 36:442-53.

Systemic glucocorticoid administration can be used alone or in addition to topical glucocorticoids for the treatment of uveitis. However, prolonged exposure to high plasma concentrations (administration of 1 mg/kg/day for 2-3 weeks) of steroid is often necessary so that therapeutic levels can be achieved in the eye.

Unfortunately, these high drug plasma levels commonly lead to systemic side effects such as hypertension, hyperglycemia, increased susceptibility to infection, peptic ulcers, psychosis, and other complications. Cheng C-K et al. (1995), Intravitreal sustained-release dexamethasone device in the treatment of experimental uveitis, Invest. Opthalmol. Vis. Sci. 36:442-53; Schwartz, B., (1966) The response of ocularpressure to corticosteroids, Opthalmol. Clin. North Am. 6:929-89; Skalka, H. W. et al., (1980), Effect of corticosteroids on cataract formation, Arch Opthalmol 98:1773-7; and Renfro, L. et al. (1992), Ocular effects of topical and systemic steroids, Dermatologic Clinics 10:505-12.

Additionally, delivery to the eye of a therapeutic amount of an active agent can be difficult, if not impossible, for drugs with short plasma half-lives since the exposure of the drug to intraocular tissues is limited. Therefore, a more efficient way of delivering a drug to treat a posterior ocular condition is to place the drug directly in the eye, such as directly into the vitreous. Maurice, D. M. (1983) Micropharmaceutics of the eye, Ocular Inflammation Ther. 1:97-102; Lee, V. H. L. et al. (1989), Drug delivery to the posterior segment” Chapter 25 In Retina. T. E. Ogden and A. P. Schachat eds., St. Louis: CV Mosby, Vol. 1, pp. 483-98; and Olsen, T. W. et al. (1995), Human scleral permeability: effects of age, cryotherapy, transscleral diode laser, and surgical thinning, Invest. Opthalmol. Vis. Sci. 36:1893-1903.

Techniques such as intravitreal injection of a drug have shown promising results, but due to the short intraocular half-life of active agent, such as glucocorticoids (approximately 3 hours), intravitreal injections must be frequently repeated to maintain a therapeutic drug level. In turn, this repetitive process increases the potential for side effects such as retinal detachment, endophthalmitis, and cataracts. Maurice, D. M. (1983), Micropharmaceutics of the eye, Ocular Inflammation Ther. 1:97-102; Olsen, T. W. et al. (1995), Human scleral permeability: effects of age, cryotherapy, transscleral diode laser, and surgical thinning, Invest. Opthalmol. Vis. Sci. 36:1893-1903; and Kwak, H. W. and D′Amico, D. J. (1992), Evaluation of the retinal toxicity and pharmacokinetics of dexamethasone after intravitreal injection, Arch. Opthalmol. 110:259-66.

Additionally, topical, systemic, and periocular glucocorticoid treatment must be monitored closely due to toxicity and the long-term side effects associated with chronic systemic drug exposure sequelae. Rao, N. A. et al. (1997), Intraocular inflammation and uveitis, In Basic and Clinical Science Course, Section 9 (1997-1998) San Francisco: American Academy of Opthalmology, pp. 57-80, 102-103, 152-156; Schwartz, B. (1966), The response of ocular pressure to corticosteroids, Opthalmol Clin North Am 6:929-89; Skalka, H. W. and Pichal, J. T. (1980), Effect of corticosteroids on cataract formation, Arch Opthalmol 98:1773-7; Renfro, L and Snow, J. S. (1992), Ocular effects of topical and systemic steroids, Dermatologic Clinics 10:505-12; Bodor, N. et al. (1992), A comparison of intraocular pressure elevating activity of loteprednol etabonate and dexamethasone in rabbits, Current Eye Research 11:525-30.

Known drug delivery systems which are placed in the vitreous or on the sclera are usually sutured in place at the sclera or have some attachment means to retain them in place so as to prevent them from becoming extruded or otherwise migrating from the original site due to the normal frequent movement of the eye. Extrusion can result in the drug delivery system eroding through the conjunctiva and being lost. Migration of the drug delivery system from its administration site can have the undesirable effect of either a suboptimal amount or an excessive amount of the therapeutic agent now reaching the target tissue.

An intraocular drug delivery system can be made of a biodegradable polymeric such as a poly(lactide) (PLA) polymers, poly(lactide-co-glycolide) (PLGA) polymers, as well as copolymers of PLA and PLGA polymers. PLA and PLGA polymers degrade by hydrolysis, and the degradation products, lactic acid and glycolic acid, are metabolized into carbon dioxide and water.

Drug delivery systems have been formulated with various active agents. For example, it is known to make 2-methoxyestradiol poly lactic acid polymer implants (as rods and wafers), intended for intraocular use, by a melt extrusion method. See eg published U.S. patent application 20050244471. Additionally, it is known to make brimonidine poly lactic acid polymer implants and microspheres intended for intraocular use. See eg published U.S. patent applications 20050244463 and 20050244506, and U.S. patent application Ser. No. 11/395,019. Furthermore, it is known to make bimatoprost containing polylactic acid polymer implants and microspheres intended for intraocular use. See eg published U.S. patent applications 2005 0244464 and 2006 0182781, and U.S. patent application Ser. Nos. 11/303,462, and; 11/371,118.

U.S. Pat. No. 6,217,895 discusses a method of administering a corticosteroid to the posterior segment of the eye, but does not disclose a bioerodible implant. U.S. Pat. No. 5,501,856 discloses controlled release pharmaceutical preparations for intraocular implants to be applied to the interior of the eye after a surgical operation for disorders in retina/vitreous body or for glaucoma. U.S. Pat. No. 5,869,079 discloses combinations of hydrophilic and hydrophobic entities in a biodegradable sustained release implant, and describes a polylactic acid polyglycolic acid (PLGA) copolymer implant comprising dexamethasone. As shown by in vitro testing of the drug release kinetics, the 100-120 μg 50/50 PLGA/dexamethasone implant disclosed did not show appreciable drug release until the beginning of the fourth week, unless a release enhancer, such as HPMC was added to the formulation.

U.S. Pat. No. 5,824,072 discloses implants for introduction into a suprachoroidal space or an avascular region of the eye, and describes a methylcellulose (i.e. non-biodegradable) implant comprising dexamethasone. WO 9513765 discloses implants comprising active agents for introduction into a suprachoroidal or an avascular region of an eye for therapeutic purposes. U.S. Pat. Nos. 4,997,652 and 5,164,188 disclose biodegradable ocular implants comprising microencapsulated drugs, and describes implanting microcapsules comprising hydrocortisone succinate into the posterior segment of the eye.

U.S. Pat. No. 5,164,188 discloses encapsulated agents for introduction into the suprachoroid of the eye, and describes placing microcapsules and plaques comprising hydrocortisone into the pars plana. U.S. Pat. Nos. 5,443,505 and 5,766,242 discloses implants comprising active agents for introduction into a suprachoroidal space or an avascular region of the eye, and describes placing microcapsules and plaques comprising hydrocortisone into the pars plana.

Zhou et al. disclose a multiple-drug implant comprising 5-fluorouridine, triamcinolone, and human recombinant tissue plasminogen activator for intraocular management of proliferative vitreoretinopathy (PVR). Zhou, T, et al. (1998), Development of a multiple-drug delivery implant for intraocular management of proliferative vitreoretinopathy, Journal of Controlled Release 55: 281-295.

U.S. Pat. No. 6,046,187 discusses methods and compositions for modulating local anesthetic by administering one or more glucocorticosteroid agents before, simultaneously with or after the administration of a local anesthetic at a site in a patient. U.S. Pat. No. 3,986,510 discusses ocular inserts having one or more inner reservoirs of a drug formulation confined within a bioerodible drug release rate controlling material of a shape adapted for insertion and retention in the sac of the eye, which is indicated as being bounded by the surfaces of the bulbar conjuctiva of the sclera of the eyeball and the palpebral conjunctiva of the eyelid, or for placement over the corneal section of the eye.

U.S. Pat. No. 6,369,116 discusses an implant with a release modifier inserted in a scleral flap. EP 0 654256 discusses use of a scleral plug after surgery on a vitreous body, for plugging an incision. U.S. Pat. No. 4,863,457 discusses the use of a bioerodible implant to prevent failure of glaucoma filtration surgery by positioning the implant either in the subconjunctival region between the conjunctival membrane overlying it and the sclera beneath it or within the sclera itself within a partial thickness sclera flap.

EP 488 401 discusses intraocular implants, made of certain polylactic acids, to be applied to the interior of the eye after a surgical operation for disorders of the retina/vitreous body or for glaucoma. EP 430539 discusses use of a bioerodible implant which is inserted in the suprachoroid.

U.S. application Ser. No. 11/565,917 filed Dec. 1, 2006 discloses intraocular (including sub-tenon's) administration of various solid, drug-containing implants.

Intraocular drug delivery systems which are sutured or fixed in place are known. Suturing or other fixation means requires sensitive ocular tissues to be in contact with aspects of a drug delivery system which are not required in order to contain a therapeutic agent within or on the drug delivery system or to permit the therapeutic agent to be released in vivo. As such suturing or eye fixation means a merely peripheral or ancillary value and their use can increase healing time, patient discomfort and the risk of infection or other complications.

### SUMMARY

The present invention provides drug delivery systems for the treatment of various ocular conditions.

### DEFINITIONS

The terms below are defined to have the following meanings:

“About” means approximately or nearly and in the context of a numerical value or range set forth herein means ±10% of the numerical value or range recited or claimed.

“Active agent”, “drug” and “therapeutic agent” are used interchangeably herein and refer to any substance used to treat an ocular condition.

“Anterior intraocular location” or “anterior ocular location” means a sub-Tenon, subchoroidal, suprachoroidal, intrascleral, episcleral, and the like intraocular location which is located no more than about 10 mm (preferably no more than about 8 mm) along the curvature of the surface of the eye from the corneal limbus.

“Biocompatible” with regard to a drug delivery system means that upon intraocular administration of the drug delivery system to a mammalian eye a significant immunogenic reaction does not occur.

“Bioerodible polymer” means a polymer which degrades in vivo. The polymer can be a gel or hydrogel type polymer. Drug delivery systems containing bioerodible polymers can have a triphasic pattern of drug release: an initial burst from surface bound drug; the second phase from diffusional release, and; release due to degradation of the polymer matrix. Thus, erosion of the polymer over time is required to release all of the active agent. The words “bioerodible” and “biodegradable” are synonymous and are used interchangeably herein.

“Drug delivery system” means a liquid, gel, hydrogel or high viscosity formulation from which a therapeutic amount of a therapeutic agent can be released upon in vivo administration of the drug delivery system. The drug delivery system is not a solid implant, although it can contain solid drug particles, microspheres and the like.

“Injury” or “damage” are interchangeable and refer to the cellular and morphological manifestations and symptoms resulting from an inflammatory-mediated condition, such as, for example, inflammation.

“Intraocular” means within or under an ocular tissue. An Intraocular administration of a drug delivery system includes administration of the drug delivery system to a sub-Tenon, subconjunctival, suprachoroidal, intravitreal and like locations. An Intraocular administration of a drug delivery system excludes administration of the drug delivery system to a topical, systemic, intramuscular, subcutaneous, intraperitoneal, and the like location.

“Ocular condition” means a disease, aliment or condition which affects or involves the eye or one or the parts or regions of the eye, such as a retinal disease. The eye includes the eyeball and the tissues and fluids which constitute the eyeball, the periocular muscles (such as the oblique and rectus muscles) and the portion of the optic nerve which is within or adjacent to the eyeball.

“Plurality” means two or more.

“Posterior ocular condition” means a disease, ailment or condition which affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerve which vascularize or innervate a posterior ocular region or site.

“Steroidal anti-inflammatory agent” and “glucocorticoid” are used interchangeably herein, and are meant to include steroidal agents, compounds or drugs which reduce inflammation when administered at a therapeutically effective level.

“Substantially” in relation to the release profile or the release characteristic of an active agent from a bioerodible implant as in the phrase “substantially continuous rate” of the active agent release rate from the implant means, that the rate of release (i.e. amount of active agent released/unit of time) does not vary by more than 100%, and preferably does not vary by more than 50%, over the period of time selected (i.e. a number of days). “Substantially” in relation to the blending, mixing or dispersing of an active agent in a polymer, as in the phrase “substantially homogenously dispersed” means that there are no or essentially no particles (i.e. aggregations) of active agent in such a homogenous dispersal.

“Suitable for insertion (or implantation) in (or into) an ocular region or site” with regard to an implant, means an implant which has a size (dimensions) such that it can be inserted or implanted without causing excessive tissue damage and without unduly physically interfering with the existing vision of the patient into which the implant is implanted or inserted.

“Sustained” as in “sustained period” or “sustained release” means for a period of time greater than thirty days, preferably for at least 20 days (i.e. for a period of time from 20 days to 365 days), and most preferably for at least 30 days. A sustained release can persist for a year or more.

“Therapeutic levels” or “therapeutic amount” means an amount or a concentration of an active agent that has been locally delivered to an ocular region that is appropriate to safely treat an ocular condition so as to reduce or prevent a symptom of an ocular condition.

Our invention encompasses a method for treating an ocular condition by preparing a biocompatible drug delivery system comprising a drug and a polymeric vehicle for the drug, and injecting the drug delivery system into an intraocular location. At least a portion of the drug remains at the intraocular location for at least about twice as long as a portion of the same drug in an aqueous vehicle injected to the same intraocular location. The polymeric vehicle can be a hydroxypropylmethylcellulose or a hyaluronic acid. The intraocular location can be an anterior intraocular location and the ocular condition can be a posterior ocular condition. A detailed embodiment of our invention is a method for treating a posterior ocular condition by preparing a biocompatible drug delivery system comprising a drug and a polymeric hyaluronic acid, and injecting the drug delivery system into an anterior intraocular location, wherein at least a portion of the drug remains at the anterior intraocular location for at least about twice as long as a portion of the same drug in an aqueous vehicle injected to the same anterior intraocular location.

Another aspect of our invention is a method for treating a posterior ocular condition by preparing a biocompatible drug delivery system comprising a drug and a polymeric hyaluronic acid, and injecting the drug delivery system into an anterior intraocular location, wherein at least a portion of the drug delivery system migrates from the anterior intraocular location to a posterior intraocular location, thereby treating the posterior ocular condition. Preferably, the drug delivery system is injected into a superior quadrant of the eye and the hyaluronic acid is a cross-linked hyaluronic acid. The intraocular location can be a sub-tenon, subconjunctival, suprachoroidal, intrascleral or retrobulbar intraocular locations.

Our invention encompasses a drug delivery system for treating an ocular condition, the drug delivery system can comprise: (a) at least one bioerodible polymer suitable for insertion into an ocular region or site, the bioerodible drug delivery system comprising; (i) an active agent, and; (ii) a bioerodible polymer, wherein the bioerodible implant can release a therapeutic level of the active agent into the ocular region or site for a period time between about 3 hours and about 1 year. Preferably, the bioerodible polymer can release the therapeutic level of the active agent into the ocular region or site at a substantially continuous rate in vivo. More preferably, the bioerodible polymer can release a therapeutic level of the active agent into the ocular region or site at a substantially continuous rate upon implantation in the vitreous for a period time between about 2 hours and about 1 year. The active agent can be an anti-inflammatory agent. The bioerodible polymer can be a PLGA co-polymer.

A bioerodible implant for treating a ocular condition can also be made as (a) a dispersion comprising an active agent dispersed with a first bioerodible polymer, (b) a particle comprising the active agent and a second bioerodible polymer, wherein the particle has an active agent release characteristic which differs from the active agent release characteristic of the dispersion. A method for treating an ocular condition according to our invention can comprise injecting into an ocular region or site a drug delivery system set forth herein.

Therapeutic agents particularly useful for inclusion in an intraocular drug delivery system for administering to an intraocular location, such as an anterior sub-Tenon's area, include antihypertensive drugs such as brimonidine tartrate, brimonidine free base, latanoprost, bimatoprost and it's analogues, beta blockers, carbonic anhydrase inhibitors, and prostaglandin receptor agonists including EP2 and EP4 E-compounds and timolol maleate. Additionally, the drug delivery system can comprise a glucocorticoid receptor blocker (such as RU-486) to help reduce corticosteroid induced ocular hypertension, as well as a sclera penetrant enhancer, such as BAK, as an excipient (especially advantageous in a sub-Tenon's implant) which acts to facilitate transit of the therapeutic agent through the sclera (i.e. by reducing the diffusion coefficient of the therapeutic agent).

### DRAWINGS

The patent or application file contains at least one drawing executed in color. Copies of this patent or patent application publication with color drawing(s) will be provided by the Office upon request and payment of the necessary fee.

FIG. 1 is photograph showing sub-Tenon's injection at the limbus of a rat eye of a polymeric formulation containing a drug surrogate, as set forth in Example 1.

FIG. 2 is a photograph of the same eye in FIG. 1 after the injection showing that a small conjunctival bleb is present post-injection.

FIG. 3 is a magnetic resonance (MR) image showing that the Gd signal in the upper portion of the sclera at 77 minutes following sub-tenon injection with the PBS only formulation (Example 1). The arrow shows that there is only a faint signal present in the sub-Tenon's space injection site.

FIG. 4 is a MR image taken at a similar time point as the image shown in FIG. 3 demonstrating a higher signal intensity (arrow) at the sub-tenon depot site using a HPMC-based formulation (Example 1) indicating a longer drug residence time. A relative high signal is noted in the aqueous humor (AH)

FIG. 5 is a MR image showing the posterior migration of the non-crosslinked HA-based formulation (indicated as “T”) (Example 1) from the anterior sub-Tenon's area (arrow shown at the place of injection) to the back of the eye.

FIG. 6 is a MR image showing a HA-based crosslinked formulation (Example 1) after the posterior migration with a final position at the posterior episcleral region overlying the posterior retinal region (indicated by the arrow). AH is the aqueous humor.

### DESCRIPTION

Our invention is based upon the discovery of particular drug delivery system formulations and methods for administering these drug delivery systems for treating various ocular conditions with increased intraocular drug residency time. The present invention encompasses drug delivery systems which are structured and configured solely for intraocular, as opposed to topical or systemic, administration. The intraocular administration can be by implantation or injection. The drug delivery systems within the scope of our invention can be biodegradable implants and microspheres. The drug delivery systems can be monolithic, that is the active agent is homogenously distributed or dispersed throughout the biodegradable polymer. The therapeutic agent can be released from drug delivery systems made according to the present invention for a period of time between about 2 hours to 12 months or more. An important feature of our drug delivery systems is that they do not include any means (such as a cap, protrusion or suture tab) for fixing the drug delivery system to the intraocular location to which it is administered.

The anterior sub-Tenon, anterior suprachoroidal space and anterior intrascleral locations extend from the corneal limbus (the location where the cornea meets the sclera) to approximately 2 mm to 10 mm posteriorly along the surface of the human eye. Further than about 10 mm from the corneal limbus one encounters posterior sub-Tenon, posterior suprachoroidal space and posterior intrascleral locations.

Significantly, we have found that administration of a suitably configured drug delivery system to an anterior intraocular location using a suitable applicator for the drug delivery system provides a self-healing method, that is not only is suturing not required to retain the drug delivery system in place, nor is suturing (stitching) always required to close the wound at the site of entry to the intraocular administration site to permit it to heal.

Our invention requires an understanding of ocular morphology and structure. The exterior surface of the globe mammalian eye can have a layer of tissue known as Tenon's capsule, underneath which lies the sclera, followed by the choroid. Between Tenon's capsule and the sclera is a virtual space known as a sub-Tenon space. Another virtual space lies between the sclera and the choroid, referred to as the suprachoroidal space. Delivery of a therapeutic agent to an ocular location the front of the eye (such as the ciliary body) can be facilitated by placement of a suitably configured drug delivery system to a location such as the anterior sub-Tenon space, the anterior suprachoroidal space. Additionally, a drug delivery system can be administered within the sclera, for example to an anterior intrascleral location. Upon lateral movement of the therapeutic agent from such drug delivery implant locations it can diffuse or be transported through the conjunctiva and sclera to the cornea. Upon perpendicular movement of the therapeutic agent through the sclera and/or the choroid it can be delivered to anterior structures of the eye. For example, an aqueous humor suppressant for the treatment of ocular hypertension or glaucoma, can be delivered from drug delivery systems placed in the anterior sub-Tenon space, the suprachoroidal space or intrascleral to the region of the ciliary body.

As can be understood an intrascleral administration of a drug delivery system does not place the drug delivery system as close to the vitreous as does a suprachoroidal (between the sclera and the choroid) administration. For that reason an intrascleral administration of a drug delivery system can be preferred over a suprachoroidal administration so as to reduce the possibility of inadvertently accessing the vitreous upon administration of the drug delivery system.

Additionally, since the lymphatic network resides in or above the tenon's fascia of the eye and deeper ocular tissues have a reduced blood flow velocity, administration of a drug delivery system in a sub-tenon and more eye interior location can provide the dual advantages of avoiding the rapid removal of the therapeutic agent by the ocular lymphatic system (reduced lymphatic drainage) and the presence of only a low circulatory removal of the therapeutic agent from the administration site. Both factors favor passage of effective amounts of the therapeutic agent to the ciliary body and trabecular meshwork target tissue.

An important characteristic of a drug delivery system within the scope of our invention is that it can be implanted or injected into an intraocular location (such as an anterior sub-Tenon, subconjunctival or suprachoroidal location) to provide sustained release of a therapeutic agent without the occurrence of or the persistence of significant immunogenicity at and adjacent to the site of the intraocular implantation or injection.

Polylactide (PLA) polymers exist in 2 chemical forms, poly(L-lactide) and poly(D,L-lactide). The pure poly(L-lactide) is regioregular and therefore is also highly crystalline, therefore degrades in vivo at a very slow rate. The poly(D,L-lactide) is regiorandom which leads to more rapid degradation in vivo. Therefore a PLA polymer which is a mixture of predominantly poly(L-lactide) polymer, the remainder being a poly(D-lactide) polymer will degrade in vivo at a rate slower that a PLA polymer which is predominantly poly(D-lactide) polymer. A PLGA is a co-polymer that combines poly(D,L-lactide) with poly(glycolide) in various possible ratios. The higher the glycolide content in a PLGA the faster the polymer degradation.

In one embodiment of our invention, a drug delivery system for intraocular administration (i.e. by implantation in the sub-Tenon space) comprises configured, consists of, or consists essentially of at least a 75 weight percent of a PLA and no more than about a 25 weight percent of a poly(D,L-lactide-co-glycolide) polymer.

The ciliary body region does not show a rapid rate of drug clearance. Hence we postulate that a therapeutic agent administered by an intraocular administration, such as by a subconjunctival injection, at the equator of the eye can from that location enter the eye to reach the ciliary body region. We selected the anterior sub-Tenon space as a preferred location for administration of a drug delivery system because from this location a therapeutic agent released from a drug delivery system we would expect to diffuse to or be transported to the ciliary body region (the target tissue). In other words, administration of a drug delivery system to the anterior sub-Tenon space can efficiently deliver an aqueous humor (elevated IOP) suppressants to the ciliary body region to treat ocular conditions such as ocular hypertension and glaucoma. For the purpose of our invention we define the anterior sub-Tenon, anterior suprachoroidal space and anterior intrascleral locations to extend from the corneal limbus (the location where the cornea meets the sclera) to approximately 2 to 10 mm posteriorly along the surface of the human eye. The ideal destination for aqueous humor suppressants entering through this region is the nonpigmented ciliary epithelium where the aqueous humor in produced. Other tissues that would be accessed with a drug delivery system in an anterior intraocular (such as sub-Tenon's) location can be the ciliary body stroma, iris root, and the trabecular meshwork. Therapeutic agents which reduce intraocular pressure primarily by improving uveoscleral flow, such as the prostamides and prostaglandins, would be efficiently delivered with a delivery system in the anterior sub-Tenon's area.

Typically, what occurs with eye drops is the active agent goes through the cornea, is fairly equally distributed through the aqueous humor, goes through the trabecular meshwork and also into the ciliary body. This all occurs 360 degrees around the eye where the ciliary body (aqueous production area) and the trabecular meshwork & iris root (where drainage occurs). Surprisingly we have determined using drug diffusion MRI imaging studies that with sub-Tenon's implants in one quadrant of the eye, the active agent drug preferentially goes through the ciliary body region in the quadrant of the implant, then the active agent goes into the aqueous humor and is equally distributed, then the active agent exits with the normal pathways of drainage (trabecular meshwork & iris root) 360 degrees Therefore, an anterior sub-Tenon's implant placed in one quadrant, can distribute active agent 360 degrees in the anterior segment.

Preferred drug delivery systems are sustained-release drug delivery systems with or without a microsphere constituent. In the adult human, the ciliary body extends 1 to 3 mm behind the corneal limbus; therefore the ideal location of the drug delivery system would 2 to 6 mm behind the limbus. Any location 360 degrees around the eye for anterior sub-Tenon's placement is permissible with the caveat that a location under the eyelid may be preferred to make the delivery system less visually apparent by others. Drug delivery systems within the scope of our invention can be placed anteriorly in the eye over the ciliary body region with an intrascleral, suprachoroidal, or intravitreal location.

Within the scope of our invention are suspensions of microspheres which can be administered to an intraocular location through a syringe needle. Administration of such a suspension requires that the viscosity of the microsphere suspension at 20° C. be less than about 300,000 cP. The viscosity of water at 20° C. is 1.002 cP (cP is centiposie, a measure of viscosity). The viscosity of olive oil is 84 cP, of castor oil 986 P and of glycerol 1490 cP The drug delivery systems of our invention can include a therapeutic agent mixed with or dispersed within a biodegradable polymer. The drug delivery systems compositions can vary according to the preferred drug release profile, the particular active agent used, the ocular condition being treated, and the medical history of the patient. Therapeutic agents which can be used in our drug delivery systems include, but are not limited to (either by itself in a drug delivery system within the scope of the present invention or in combination with another therapeutic agent): ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCI; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, azathioprine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; beta.-adrenergic blocker or beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), timolol, mebefunolol; memantine, latanoprost (Xalatan); alpha-2 adrenergic receptor agonists; 2-methoxyestradiol; anti-neoplastics, such as vinblastine, vincristine, interferons; alpha, beta and gamma, antimetabolites, such as folic acid analogs, purine analogs, and pyrimidine analogs; immunosuppressants such as azathiprine, cyclosporine and mizoribine; miotic agents, such as carbachol, mydriatic agents such as atropine, protease inhibitors such as aprotinin, camostat, gabexate, vasodilators such as bradykinin, and various growth factors, such epidermal growth factor, basic fibroblast growth factor, nerve growth factors, carbonic anhydrase inhibitors, and the like.

In particular embodiments of our invention, the active agent can be a compound that blocks or reduces the expression of VEGF receptors (VEGFR) or VEGF ligand including but not limited to anti-VEGF aptamers (e.g. Pegaptanib), soluble recombinant decoy receptors (e.g. VEGF Trap), anti-VEGF monoclonal antibodies (e.g. Bevacizamab) and/or antibody fragments (e.g. Ranibizamab), small interfering RNA's decreasing expression of VEGFR or VEGF ligand, post-VEGFR blockade with tyrosine kinase inhibitors, MMP inhibitors, IGFBP3, SDF-1 blockers, PEDF, gamma-secretase, Delta-like ligand 4, integrin antagonists, HIF-1 alpha blockade, protein kinase CK2 blockade, and inhibition of stem cell (i.e. endothelial progenitor cell) homing to the site of neovascularization using vascular endothelial cadherin (CD-144) and stromal derived factor (SDF)-1 antibodies.

In another embodiment or variation of our invention the active agent is methotrexate. In another variation, the active agent is a retinoic acid. In another variation, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent, such as dexamethasone.

Steroidal anti-inflammatory agents that can be used in our drug delivery systems can include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one embodiment, cortisone, dexamethasone, fluocinolone, hydrocortisone, methylprednisolone, prednisolone, prednisone, and triamcinolone, and their derivatives, are preferred steroidal anti-inflammatory agents. In another preferred variation, the steroidal anti-inflammatory agent is dexamethasone. In another variation, the biodegradable implant includes a combination of two or more steroidal anti-inflammatory agents.

The active agent, such as a steroidal anti-inflammatory agent, can comprise from about 10% to about 90% by weight of the implant. In one variation, the agent is from about 40% to about 80% by weight of the implant. In a preferred variation, the agent comprises about 60% by weight of the implant. In a more preferred embodiment of the present invention, the agent can comprise about 50% by weight of the implant.

The therapeutic active agent present in our drug delivery systems can be homogeneously dispersed in the biodegradable polymer of the drug delivery system. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active agent of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the drug delivery system.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, Biodegradable Polymers in Controlled Drug Delivery, In: “CRC Critical Reviews in Therapeutic Drug Carrier Systems”, Vol. 1. CRC Press, Boca Raton, Fla. (1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Other agents may be employed in a drug delivery system formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable drug delivery systems can also include additional hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

A drug delivery system within the scope of the present invention can be formulated with particles of an active agent dispersed within a biodegradable polymer. Without being bound by theory, it is believed that the release of the active agent can be achieved by erosion of the biodegradable polymer matrix and by diffusion of the particulate agent into an ocular fluid, e.g., the vitreous, with subsequent dissolution of the polymer matrix and release of the active agent. Factors which influence the release kinetics of active agent from the implant can include such characteristics as the size and shape of the implant, the size of the active agent particles, the solubility of the active agent, the ratio of active agent to polymer(s), the method of manufacture, the surface area exposed, and the erosion rate of the polymer(s).

The release rate of the active agent can depend at least in part on the rate of degradation of the polymer backbone component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of active agent release.

The release kinetics of the drug delivery systems of the present invention can be dependent in part on the surface area of the drug delivery systems. A larger surface area exposes more polymer and active agent to ocular fluid, causing faster erosion of the polymer and dissolution of the active agent particles in the fluid.

Examples of ocular conditions which can be treated by the drug delivery systems and methods of the invention include, but are not limited to, glaucoma, uveitis, macular edema, macular degeneration, retinal detachment, posterior ocular tumors, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, and vascular occlusion. In one variation, the implants are particularly useful in treating such medical conditions as uveitis, macular edema, vascular occlusive conditions, proliferative vitreoretinopathy (PVR), and various other retinopathies.

The drug delivery systems of our invention can be injected to an intraocular location by syringe or can be inserted (implanted) into the eye by a variety of methods, including placement by forceps, by trocar, or by other types of applicators, after making an incision in the sclera. In some instances, a trocar or applicator may be used without creating an incision. In a preferred variation, a hand held applicator is used to insert one or more biodegradable implants into the eye. The hand held applicator typically comprises an 18-30 GA stainless steel needle, a lever, an actuator, and a plunger. Suitable devices for inserting an implant or implants into a posterior ocular region or site includes those disclosed in U.S. patent application Ser. No. 10/666,872.

The method of administration generally first involves accessing the target area within the ocular region with the needle, trocar or implantation device. Once within the target area, e.g., the vitreous cavity, a lever on a hand held device can be depressed to cause an actuator to drive a plunger forward. As the plunger moves forward, it can push the implant or implants into the target area (i.e. the vitreous).

Various techniques may be employed to make implants within the scope of the present invention. Useful techniques include phase separation methods, interfacial methods, extrusion methods, compression methods, molding methods, injection molding methods, heat press methods and the like.

The drug delivery systems disclosed herein can be used to prevent or to treat various ocular diseases or conditions, including the following: maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

### EXAMPLE

The following example illustrate aspects and embodiments of our invention.

### Example 1

### Polymeric Formulations Providing Increased Intraocular Drug Residency Time

Summary

In this experiment we made and tested polymeric, drug surrogate containing formulations which when administered to an anterior intraocular location, such as the sub-tenon space, provided an increased time of intraocular (i.e. sub-tenon) residency of the drug surrogate, as compared the intraocular residency time of the same drug surrogate injected at the same intraocular location in a non-polymeric (i.e. aqueous) formulation. We also determined that anterior intraocular administration of a drug surrogate in a polymeric vehicle, such as particular high viscosity depot vehicles (such as hyaluronic acid) is an effective means for transporting both the depot vehicle and the drug surrogate contained therein to a posterior ocular location for (upon substitution of the drug surrogate for a small molecular weight therapeutic agent) effectively treating a posterior ocular condition. For convenience in this Example the terms “drug surrogate” and “drug” are used synonymously.

These observations are surprising because it was expected that intraocular administration of drug in a polymeric vehicle would led to either immediate (i.e. within a few minutes) burst release of the drug or release over an extended period (i.e. over weeks or months) of intermittent dug release, as opposed to the observed drug release from the polymeric carrier over several hours. Additionally, it was unexpected that (anterior intraocular administered) polymeric vehicle with significant amounts of drug contained therein would by some unknown transport mechanism migrate to the back of the eye.

Introduction

This experiment was carried out to determine if particular polymeric hydrogel formulations can increase small molecule (i.e. molecular weight less than about 2,000 Daltons) drug residency times when injected into an intraocular site, such as the sub-Tenon's space. A hydrogel is a colloidal gel formed as a dispersion in water or other aqueous medium. Thus a hydrogel is formed upon formation of a colloid in which a dispersed phase (the colloid) has combined with a continuous phase (i.e. water) to produce a viscous jellylike product; for example, coagulated silicic acid. A hydrogel is a three-dimensional network of hydrophilic polymer chains that are crosslinked through either chemical or physical bonding. Because of the hydrophilic nature of the polymer chains, hydrogels absorb water and swell. The swelling process is the same as the dissolution of non-crosslinked hydrophilic polymers. By definition, water constitutes at least 10% of the total weight (or volume) of a hydrogel.

Examples of hydrogels include synthetic polymers such as polyhydroxy ethyl methacrylate, and chemically or physically crosslinked polyvinyl alcohol, polyacrylamide, poly(N-vinyl pyrolidone), polyethylene oxide, and hydrolysed polyacrylonitrile. Examples of hydrogels which are organic polymers include covalent or ionically crosslinked polysaccharide-based hydrogels such as the polyvalent metal salts of alginate, pectin, carboxymethyl cellulose, heparin, hyaluronate and hydrogels from chitin, chitosan, pullulan, gellan and xanthan. The particular hydrogels used in our experiment were a cellulose compound (i.e. hydroxypropylmethylcellulose [HPMC]) and a high molecular weight hyaluronic acid (HA).

The clearance of small molecular weight drugs in aqueous solution from the sub-Tenon's space is rapid, generally with a half-life under 1-hour.¹ In contrast, the same aqueous solution drugs have a vitreous half of 3 to 4 hours following an intravitreal injection.² Unfortunately, for the desired result of greater intraocular drug residency time, there is an extensive network of lymphatic and blood vessels in the conjunctiva and episclera that facilitates the clearance of drugs following a sub-Tenon's injection.³ Increasing the residency times of drugs in the sub-Tenon's space can have therapeutic value by increasing the ability of the drug to be transported to tissues within the eye, such as the aqueous humor, vitreous, and retina.

Intraocular Administration

Our formulations can be administered by injection to various intraocular sites as explained below. For example, a subconjunctival periocular administration can be carried out by elevating the bulbar conjunctiva in the superior-temporal quadrant using forceps. A 26-gauge, ½-inch hypodermic needle, with the bevel facing upward can then be inserted into the subconjunctival space and the formulation injected. Alternately, a sub-tenon's Injection can be carried out by inserting a 25-gauge, ⅝-inch hypodermic needle, with the bevel facing upwards, through the bulbar conjunctiva (2 to 3 mm above the corneal limbus) and Tenon's capsule in the superior temporal quadrant beginning 2 to 3 mm from the limbus. A small bleb can be made at this location by injecting 5 to 10 μL of sterile saline. The bleb of tissue can be punctured with a 21-gauge, 1-inch, hypodermic needle to allow insertion of a blunt cannula into the sub-Tenon's space. A 23-gauge blunt cannula can be inserted into the sub-Tenon's space and advanced in a posterior manner approximately 10 to 15 mm in a superior temporal direction. The formulation can then be injected.

Additionally, a retrobulbar injection can be carried out using a 22-gauge, 1.5-inch spinal needle curved to follow the curve of the orbit from anterior to posterior. The needle can be inserted at the conjunctival junction of the lateral canthus and the needle advanced posteriorly until the needle encounters the orbital bone at the posterior portion of the globe. The stylet is then removed and aspiration performed. If blood is aspirated, the needle can be repositioned and the aspiration performed a second time. If blood is not aspirated the formulation is then injected and the needle removed. Following each administration AKWA Tears®, a bland ophthalmic ointment can be applied to the dosed eyes of each animal to prevent the eyes from drying out and ciprofloxacin ointment applied after dosing.

Materials and Methods

Formulations containing either a HPMC or HA polymer were made. Hydroxypropylmethylcellulose (HPMC) is an ether of methyl cellulose and occurs as a white, combustible, water-soluble powder. It has many uses include applications as an emulsifier, stabilizer, and thickener in foods, and as an ophthalmic lubricant. (Synonyms for HPMC include cellulose 2-hydroxypropyl methyl ether, hypromellose, and propylene glycol ether). The HPMC used in this experiment was Methocel E4M Premium (a water-soluble cellulose ether), obtained from Colorcon, WestPoint, Pa.

The non-cross-linked HA used was a sodium hyaluronate powder purchased from Genzyme, Cambridge, Mass. as bulk hyaluronan. Typical and most useful molecular weights for this HA polymer are 1.0 to 1.9 million Daltons). The cross-linked HA used in this experiment was Juvederm® (Allergan, Irvine, Calif.). The non-crosslinked HA was constituted as a hydrogel by mixing the HA powder with the appropriate amount of water (so as to obtain the Table 1 formulation parameters) followed by mixing with Magnevist contract agent (Berlex, Wayne, N.J.). Juvederm® is already constituted as a hydrogel so it needs only to be mixed with the Magnevist. Each HA used was mixed with an amount of Magnevist so as to provide a concentration of the Magnevist contract agent in the HA hydrogel of 0.05 M (see Table 1).

The four formulations used in this experiment are shown in Table 1. In Table 1 PBS is phosphate buffered saline, HPMC is the hydroxypropylmethlcellulose used and HA is the hyaluronic acid used. Magnevist (gadopentetate dimeglumine, molecular weight 938 Daltons) (Gd) was used as a drug surrogate (i.e. representing a low molecular weight or small molecule drug) in this experiment. All four formulations were formulated to contain 0.05 M Gd. The PBS formulation comprised only PBS and the Gd contrast agent. The purpose of the PBS formulation was to serve as a control to determine how long the Gd would remain resident in the sub-tenon space where injected in an aqueous, physiological vehicle. The HPMC formulation was made using a 4 wt % HPMC solution which when combined with the Gd provided a final HPMC concentration in the formulation of 3.6 wt %. The non-crosslinked HA formulation was formulated using a 2.3 wt % HA dispersion which when combined with the Gd provided a final non-cross-linked HPMC concentration in the formulation of 2.07 wt %. The cross-linked HA formulation was formulated using a 2.4 wt % HA dispersion which when combined with the Gd provided a final cross-cross-linked HPMC concentration in the formulation of 2.4 wt %.

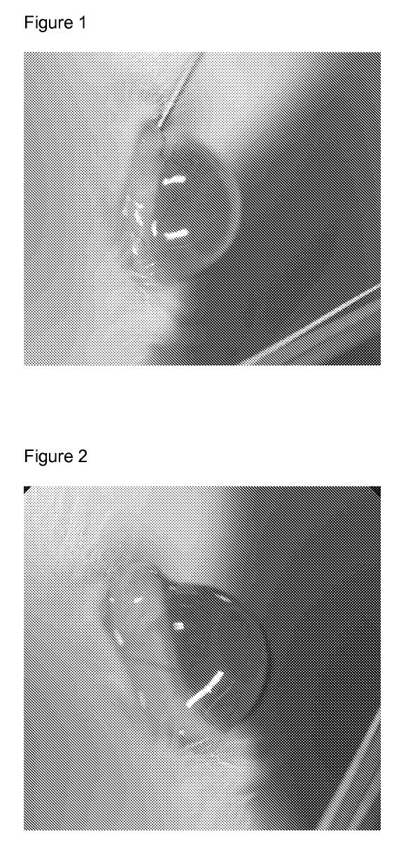

Injections were performed in Sprague Dawley rats under inhalation isoflorane anesthesia 1 mm posterior to the limbus between the 12 o'clock and 1 o'clock positions (i.e. superior quadrant eye injections) using a 30 gauge one half inch needle with a volume of 10 μL of the selected formulation injected. The needle was introduced into the sub-tenon's space approximately 1-2 mm before the injection to have a self sealing hole in the conjunctiva (see FIG. 1) and upon removal of the injection needle only a small conjunctival bleb was raised (see FIG. 2).

Selected animals had an inferior quadrant eye injection between the 5 o'clock and 6 o'clock positions to determine if there was a difference in sub-tenon's drug residency time as a function of the (superior or inferior eye quadrant) location of the injection.

Subsequently, the animals were placed in a high resolution magnetic resonance imaging MRI instrument (7T Bruker Pharmascan, Ettlingen, Germany) and sequential 3D and 2D images were obtained with a volume coil until no Gd signal was detected in the sub-tenon's space. The primary outcome measure was the detection of Gd by either 3D or 2D MRI scans, and the endpoint was the Gd (drug surrogate) sub tenon's residency time, defined as the time point at which no Gd signal was detected by the MRI in the conjunctiva, sub-Tenon's space or sclera.

TABLE 1
FORMULATIONS

| | PBS | HPMC | NON-CROSSLINKED HA | CROSSLINKED HA |
|---|---|---|---|---|
| WT % POLYMER | N/A | 4% | 2.3% | 2 TO 4% |
| FINAL GEL | N/A | 3.6% | 2.07% | 1.5 TO 3.5% |
| CONCENTRATION | | | | |
| FINAL MAGNEVIST | 0.05M | 0.05M | 0.05M | 0.05M |
| CONCENTRATION | | | | |
| VISCOSITY (CPS) | 1.10 | 41,000 | 81,000 | 25,700 |
| AT 25° C. | | | | |
| % CROSSLINKED | N/A | N/A | 0% | 85 TO 99% |

Results

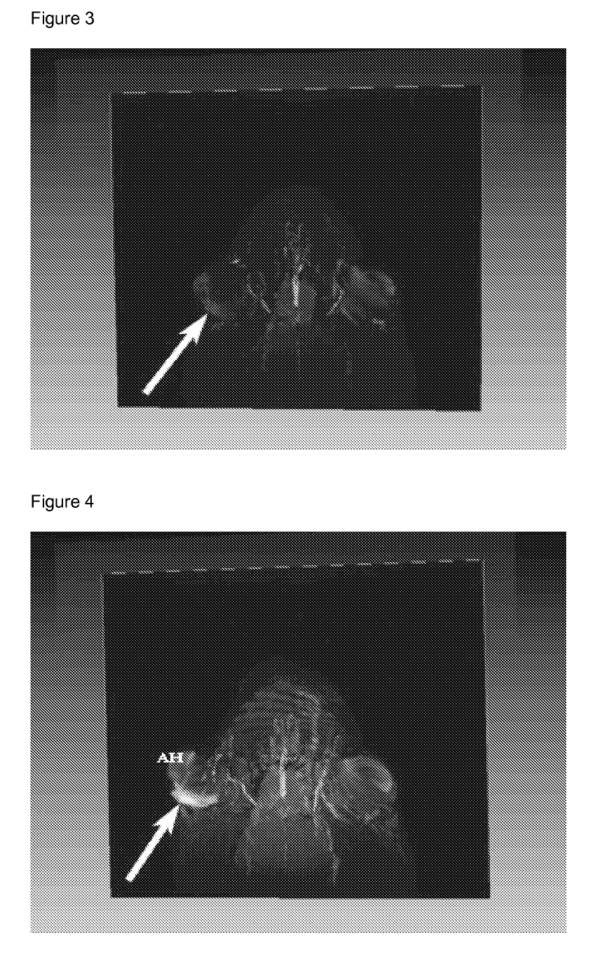

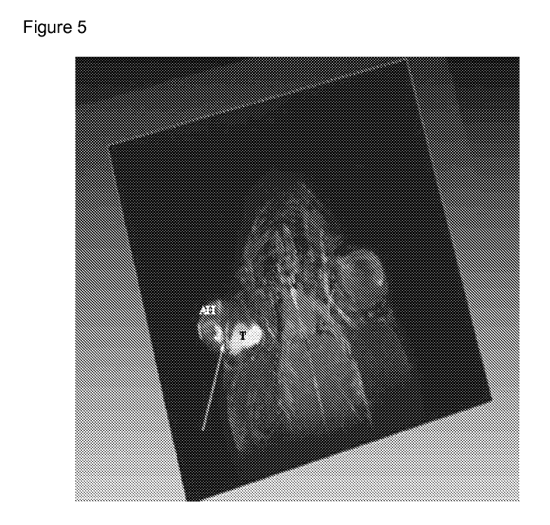

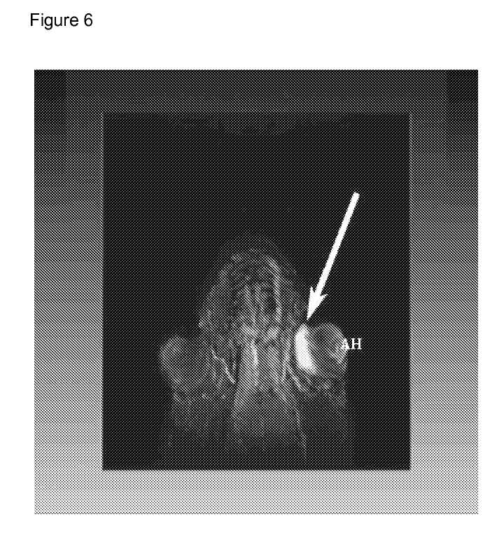

The PBS formulation showed a mean Gd residency time in the sub-tenon space of 96.3 minutes upon superior eye quadrant injection of the PBS formulation and 58 minutes upon inferior eye quadrant injection of the same PBS formulation (see Table 2 and FIG. 3). In Table 2 “N” means the number of animals injected with that formulation. Significantly, the HPMC and HA formulations increased the Gd residency time nearly 2 fold (see Table 2 and FIG. 4). Surprisingly we determined that both HA formulations migrated to the back of the eye from the anterior sub-Tenon's injection area (see FIG. 5). The cross-linked HA posterior migration was especially rapid and the polymer vehicle depots came to rest at a final position at the posterior episcleral region overlying the posterior retinal region (see FIG. 6)—a location highly significant for effective, precise dosing treatment of retinal disorders such as macular degeneration, macular edema, retinal neovascularization, and glaucoma related optic nerve degeneration. Significantly, the HA depots were still present after the Gd was absent from the depot, indicating that other drugs with a higher depot residency time can be so placed over the retina from an anterior intraocular injection.

TABLE 2
Residency Times of Magnevist in Polymeric Formulations

| | | | | Non- | Cross- |
|---|---|---|---|---|---|
| | | | | crosslinked | linked |
| | PBS | PBS | HPMC | HA | HA |
| | (superior | (inferior | (superior | (superior | (superior |
| | injection) | injection) | injection) | injection) | injection) |
| N | 3 | 2 | 1 | 2 | 2 |
| Mean | 96.3 | 58 | 179 | 176.5 | 172.5 |
| (minutes) | | | | | |
| Posterior | No | No | No | Yes | Yes |
| migration | | | | | |
| of | | | | | |
| polymer | | | | | |

### CONCLUSION

From this experiment we determined that the residency time of a drug (for example in a PBS vehicle) injected into an intraocular location, such as the sub-Tenon's space is enhanced when the injection is made into a superior quadrant of the eye. This may occur because the major lymphatic trunks exiting the conjunctival to the regional lymph nodes are located inferotemporally and inferonasally. Hence, injecting a drug depots adjacent to the exiting lymphatics inferiorly may act to reduce the drug residency as compared to a superior quadrant injections of the same drug.

We also determined that intraocular drug residency time can be enhanced in the sub-Tenon's region by use of either an HPMC or HA (non-cross linked or cross linked) formulation. Unexpectedly, a depot of a HA-based formulations, especially the crosslinked HA migrated from an anterior position on the globe to the posterior episcleral region. This newly determined carrier or vehicle migration property may be exploited to enhance trans-scleral delivery to the posterior retina using a minimally invasive anterior sub-Tenon's injection. This can avoid the requirement of a conjunctival cut-down and a curved cannula to inject into the episcleral space in the posterior region for trans-scleral delivery to the macula.

We found that the HA is present in this posterior eye location to which it has migrated even after the Gd has diffused completely from the formulation. Extrapolating with what is known with the residency of a cross-linked HA in the dermis (i.e. upon administration of Juvederm®), it can be expected that HA would remain in the episcleral space for a period of from weeks to months. This HA migration and subsequent posterior eye residency property of HA injected sub-tenon can be used advantageously with various drug formulations. For example, a sustained-release formulation such as a microsphere encapsulated drug, or sparingly soluble drug crystals, such as rapamycin, cyclosporine, or any crystalline corticosteroid (such as triamcinolone acetonide), can be formulated with the HA which will then, upon sub-tenon injection, transport the drug particles to the eye posterior, as shown in this experiment, thereby enhancing the duration of drug release to the intraocular tissues including the retina. Having the drug exposed primarily to the posterior aspect of the globe may also be advantageous when trying to avoid anterior segment drug exposure. An example is the use of a corticosteroid, such as triamcinolone acetonide, where anterior exposure can lead to corticosteroid-responsive glaucoma.

We found that the superior or upper part of the eye is a better location for formulation administration for increasing the drug concentrations into the eye, as compared to an inferior or lower eye quadrant location. The principal elimination mechanism of the conjunctiva are through the lymphatic drainage system. The lymphatics are present in a bilayer, one fine network just below the conjunctival epithelium, another layer that communicates with the other that is located in the mid-zone of the Tenon's fascia and has lymphatic vessels that are larger in diameter. The lymphatics are located diffusely around the anterior conjunctiva and drain through larger lymphatic vessels located in both the inferotemporal region and also the inferonasal region. From here, the lymphatics merge into the cervical lymph node and medial lymph node chains, respectively. They further drain inferiorly and end up in larger lymph vessels, such as the thoracic duct, and then into to venous blood system. Since the net movement of lymph fluid on the eye is from superior to inferior (from upper to lower eye surface), placing drug delivery systems on the episclera superiorly allows for greater ocular drug contact time and this can increase drug concentrations in the eye. Conversely, formulation injection inferiorly can lead to shorter contact times since the drug released is closer to the main lymphatic elimination trunks. Notably the art teaches placing a drug delivery system implant in the inferior quadrants of the eye because the lower eyelid is less likely to cause extrusion of the administered drug delivery system.

Diseases that can be treated with a drug depot located in the posterior aspect of the eye include but are not limited to posterior scleral conditions such as posterior scleritis, orbital conditions such as inflammatory orbital pseudotumor, and optic nerve diseases such as optic neuritis. In addition to HPMC and HA, other polymers (alginates, chitosans, etc.) with different Vander Waal's interactions with the drug can be used to slow the release of the drug from the hydrogel depot vehicle. Additionally, ionic interactions or hydrogen bonding can be utilized to sustain drug residence in the polymer system e.g. sodium carboxymethylcellulose and a cationic drug. Pure injectable polymers such as poly (orthoesters) can be utilized in the invention controlling release of drug by diffusion, bulk erosion or surface erosion. Compounds can also be tethered to the polymer backbone by chemically or enzymatically labile bonds. Polymers solublized in low molecular weight, diffusible solvents with drug incorporated can be utilized with this invention. Upon diffusion of the solvent from the sub-Tenon's space the drug will become entrapped in the polymer system, increasing viscosity over saline injection and achieving the benefits of this invention. Viscous liquid non-polymeric systems such as sucrose acetate isobutyrate can be utilized. Self emulsifying systems creating a vehicle with increased viscosity upon emulsification can also be used in this invention. Microparticulates, nanoparticles, nanocapsules, microcapsules and similar solid form delivery systems can be incorporated into this invention. Polymer systems that undergo phase transitions in response to various stimuli for intraocular use resulting in large volume and or viscosity change in the system can be utilized. The system can respond to pH, ionic environment, temperature, biologic triggers as well as other chemical and physical triggers. The system comprises one or more polymers capable of interacting to cause a phase-transition resulting in the volume or viscosity increases. Examples of polymers include polyacrylic acid and polyacrylamide. The drug can be physically entrapped or chemically bound via hydrogen binding, ionic interactions, van der Waals forces or hydrophobic interactions. Release of the drug can be controlled by physical entrapment of the active compound in the contracted gel. Volume expansion of the gel in response to the appropriate stimulus will facilitate diffusion of the active out of the system. For compounds that are physically or chemically bound to the polymers comprising the phase transition gel the volume expansion serves to act as a depot for drug delivery.

Drug particles can be potentially inflammatory as manifested by the clinical syndrome of sterile endophthalmitis following the injection of Kenalog into the vitreous humor.⁴ Drug particles, such as those in Kenalog, can also be inflammatory in the sub-Tenon's space. Fortunately, HA is native and has inherent anti-inflammatory properties.⁶ Therefore, when applied to the sub-Tenon's space, encapsulation of drug particles with HA can potentially reduce the incidence of corticosteroid particle induced inflammation in the conjunctival tissues.

### EXAMPLE 1 REFERENCES

1. Kim S H, Galban C J, Lutz R J, et al. Assessment of subconjunctival and intrascleral drug delivery to the posterior segment using dynamic contrast-enhanced magnetic resonance imaging. Invest Opthalmol Vis Sci 2007; 48:808-14.
2. Macha S, Mitra A K. Ocular pharmacokinetics of cephalosporins using microdialysis. J Ocul Pharmacol Ther 2001; 17:485-98.
3. Robinson M R, Lee SS, Kim H, et al. A rabbit model for assessing the ocular barriers to the transscleral delivery of triamcinolone acetonide. Exp Eye Res 2006; 82:479-87.
4. Wang L C, Yang C M. Sterile endophthalmitis following intravitreal injection of triamcinolone acetonide. Ocul Immunol Inflamm 2005; 13:295-300.
5. Giangiacomo J, Dueker D K, Adelstein E H. Histopathology of triamcinolone in the subconjunctiva. Opthalmology 1987; 94:149-53.
6. Liao Y H, Jones S A, Forbes B, Martin G P, Brown M B. Hyaluronan: pharmaceutical characterization and drug delivery. Drug Deliv 2005; 12:327-42.

All references, articles, patents, applications and publications set forth above are incorporated herein by reference in their entireties.

Accordingly, the spirit and scope of the following claims should not be limited to the descriptions of the preferred embodiments set forth above.

We claim:

1. A method for treating an ocular condition, the method comprising the steps of:
(a) preparing a biocompatible drug delivery system comprising a drug and a polymeric vehicle for the drug, and;
(b) injecting the drug delivery system into an intraocular location, wherein at least a portion of the drug remains at the intraocular location for at least about twice as long as a portion of the same drug in an aqueous vehicle injected to the same intraocular location.

2. The method of claim 1, wherein the polymeric vehicle is a hydroxypropylmethylcellulose.

3. The method of claim 1, wherein the polymeric vehicle is a hyaluronic acid.

4. The method of claim 1, wherein the intraocular location is an anterior intraocular location.

5. The method of claim 1, wherein the ocular condition is a posterior ocular condition.

6. A method for treating a posterior ocular condition, the method comprising the steps of:
(a) preparing a biocompatible drug delivery system comprising a drug and a polymeric hyaluronic acid, and;
(b) injecting the drug delivery system into an anterior intraocular location, wherein at least a portion of the drug remains at the anterior intraocular location for at least about twice as long as a portion of the same drug in an aqueous vehicle injected to the same anterior intraocular location.

7. A method for treating a posterior ocular condition, the method comprising the steps of:
(a) preparing a biocompatible drug delivery system comprising a drug and a polymeric hyaluronic acid, and;
(b) injecting the drug delivery system into an anterior intraocular location, wherein at least a portion of the drug delivery system migrates from the anterior intraocular location to a posterior intraocular location, thereby treating the posterior ocular condition.

8. The method of claim 7, wherein the drug delivery system is injected into a superior quadrant of the eye.

9. The method of claim 7 wherein the hyaluronic acid is a cross-linked hyaluronic acid.

10. The method of claim 7 wherein the hyaluronic acid is a noncross-linked hyaluronic acid.

11. A method for treating a posterior ocular condition, the method comprising the steps of:
(a) preparing a biocompatible drug delivery system comprising a drug and a polymeric hyaluronic acid, and;
(b) injecting the drug delivery system into a superior quadrant of the eye, wherein at least a portion of the drug delivery system migrates from the anterior intraocular location to a posterior intraocular location, thereby treating the posterior ocular condition.

12. A method for treating a posterior ocular condition, the method comprising the steps of:
(a) preparing a biocompatible drug delivery system comprising a liquid polymeric carrier and a drug, and;
(b) administering the drug delivery system to an anterior intraocular location thereby treating the posterior ocular condition.

13. The method of claim 12, wherein the intraocular location is selected from the group of intraocular locations consisting of sub-tenon, subconjunctival, suprachoroidal, intrascleral and retrobulbar intraocular locations.
